(19) United States
(12) Patent Application Publication
(10) Pub. No.: US 2005/0238604 A1
(43) Pub. Date: Oct. 27, 2005

(54) Cosmetic composition containing particles of elastomeric silicone

(75) Inventor: Guillaume Cassin, Villebon sur Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL S.A., Paris (FR)

(21) Appl. No.: 10/508,007

(22) PCT Filed: Mar. 17, 2003
(86) PCT No.: PCT/IB03/01428

§ 371 Date: Jun. 10, 2005

Related U.S. Application Data
(60) Provisional application No. 60/368,969, filed on Apr. 2, 2002.

(30) Foreign Application Priority Data
Mar. 25, 2002 (FR) 0203700

(51) Int. Cl. A61K 7/06; A61K 7/11

(52) U.S. Cl. 424/70.12

(57) ABSTRACT

The invention relates to a composition for topical application containing, in a physiologically acceptable medium, at least one polymer chosen from polymers capable of forming a thermoreversible gel, polymers capable of forming a crosslinked gel, and mixtures thereof, and an aqueous dispersion of particles of elastomeric organopolysiloxane. The polymers are preferably chosen from carrageenans, agars, gellan gums, alginates, pectins and mixtures thereof. The composition obtained preferably has a pasty texture and has the advantage of being both very soft and very cooling. The subject of the invention is also the uses, in particular the cosmetic uses, of the claimed composition, in particular for concealing and/or softening imperfections of the skin and/or the lips.

The present invention relates to a composition for topical application, containing a polymer capable of forming a thermoreversible or crosslinked gel, and an aqueous dispersion of particles of elastomeric organopolysiloxane. The invention also relates to the uses, in particular the cosmetic uses, of this composition, in particular for the treatment and/or care of the skin, the mucous membranes (lips), more particularly for softening, correcting and/or concealing imperfections of the skin and/or the lips.

The care compositions for the skin or the make-up compositions used for softening or concealing skin defects, such as skin microreliefs, wrinkles, fine lines, pores or colour variations, are often products having a pasty texture, generally anhydrous products containing oily gelling agents and fillers. These compositions have the disadvantage of being difficult to spread and of having a dry effect on the skin. However, the properties sought for a cosmetic composition are not only its efficacy, but also its pleasant qualities, which results, for example, in effects of softness and/or of cooling during application to human skin or any other keratinous support in human beings.

It is known to incorporate into cosmetic compositions silicone resins which give good sensory properties to these compositions during application to the skin, in particular softness. However, cosmetic compositions containing such resins have the disadvantage of not offering a cooling effect upon application to the skin. In addition, the introduction of these resins into aqueous compositions which, for their part, can offer the desired cooling effect, requires, in order to stabilize the dispersion, the use of surfactants in quantities of the order of a few per cent (generally greater than 0.5% and even greater than 1% of the total weight of the composition), which can cause problems of irritation, in particular for people with sensitive skin.

The need therefore remains for a composition for topical application, in particular a cosmetic composition, which may have a pasty texture and which is capable of softening the imperfections of the skin, while offering both softness and coolness during application to the skin, and while containing little or no emulsifier.

Surprisingly, the applicant found that it was possible to achieve the aim of the invention by combining a polymer capable of forming a thermoreversible or crosslinked gel with an aqueous dispersion of particles of an elastomeric organopolysiloxane.

Admittedly, it is known to use polymers capable of forming a thermoreversible or crosslinked gel, in cosmetic compositions. However, introducing particles of elastomeric silicone into such a gel has never been described, much less introducing an aqueous dispersion of particles of elastomeric silicone into a gel of this type containing a large quantity of polymer.

Thus, the document EP-A-355908 describes the use of polymers capable of forming reversible gels, in order to obtain stable fluid compositions, characterized by a modulus of elasticity of less than 200 Pascals. This document neither describes nor suggests the addition of an aqueous dispersion of particles of elastomeric silicone nor that such an addition makes it possible to obtain pasty compositions, offering both coolness and softness during application to the skin. The pasty compositions of the present invention are characterized by a modulus of elasticity at 25° C. greater than 5000 Pascals, and preferably greater than 10 000 Pascals, and a yield point greater than 50 Pascals and preferably greater than 100 Pascals.

The document JP-A-2000/119166 describes fluid gels obtained from polysaccharides and containing a water-soluble thickener as stabilizing agent. The stabilizing agent may be native gellan. However, at the concentrations used in this document, gellan behaves like a water-soluble thickening polymer, that is to say that it forms a rheofluidizing gel which becomes liquid when it is sheared by stirring it vigorously, but it does not form a gel for the purposes of the present invention, this gel being defined by the formation of a permanent three-dimensional network which does not liquefy when it is sheared, unlike rheofluidizing gels. Furthermore, while this document mentions generally, among a list of possible additives, silicone resins and silicone powders, the introduction of such additives takes place in the presence of surfactants (see Example 6 containing a phenylmethicone). In addition, powders of elastomeric silicone are neither described nor illustrated among these additives, much less aqueous dispersions of elastomeric silicone. In addition, the aim sought in this document is to obtain fluid gels, and nothing urges persons skilled in the art, starting with this document, to make pasty compositions.

Finally, the document WO-A-99/51193 describes cleansing and conditioning fluid compositions for the hair, containing dispersions of natural polymers, called sheared gels, and a suspended phase which may consist of conditioning agents such as weakly crosslinked silicone gums, optionally in pre-emulsified form. The silicone gums described in this document are weakly crosslinked, and the pre-emulsified dispersions described have properties for thickening oily media and are therefore different from the aqueous dispersions of highly crosslinked elastomeric organopolysiloxane which are used in the present application, and which are introduced into an aqueous phase. In addition, the introduction of these weakly crosslinked silicone gums into the hair compositions described in this document requires the use of a surfactant, which is most often charged (cationic in particular), in amounts greater than 0.5% (for example 0.7% of Arquad 16-29 in Example 2), while the compositions according to the invention preferably contain less than 0.05% by weight of surfactant relative to the total weight of the composition. Finally, the desired aim in this document is to obtain fluid compositions, which runs counter to the aim of the present application.

Thus, the documents indicated above all describe fluid compositions, whereas the aim of the present application is to obtain pasty compositions. Moreover, none of these documents either describes or suggests combining polymers giving thermoreversible or crosslinked gels with aqueous dispersions of particles of elastomeric silicone, in order to obtain compositions which may have a pasty texture, while being cooling and while containing only little or no emulsifying surfactants.

Accordingly, the invention relates to a composition containing, in a physiologically acceptable medium, (a) at least one polymer chosen from polymers capable of forming a thermoreversible gel, polymers capable of forming a crosslinked gel, and mixtures thereof, and (b) an aqueous dispersion of particles of elastomeric organopolysiloxane.

The expression “physiologically acceptable” is understood to mean a medium compatible with the skin, including the scalp, the mucous membranes (lips), the eyes and the keratinous fibres of human beings.

The expression “elastomeric” is understood to mean a soft, deformable material having viscoelastic properties and in particular the consistency of a sponge or of a soft sphere. Its modulus of elasticity is such that this material is resistant to deformation and possesses a limited capacity for extension and for contraction. This material is capable of returning to its original shape after pulling. This elastomer consists of polymeric chains of high molecular weight whose mobility is limited by a uniform network of crosslinking points. The elastomeric organopolysiloxanes in accordance with the invention are partially or completely crosslinked, and have a three-dimensional structure.

The composition according to the invention may be advantageously obtained by simply mixing in the hot state the aqueous dispersion of silicone particles and the polymer, and then cooling the mixture obtained while shearing it by vigorous stirring, as indicated in the method of preparation described below. This method makes it possible to obtain aqueous cosmetic compositions containing silicone powders, free of surfactants or of stabilizing polymers, that is to say obtained without the addition of surfactants or of stabilizing polymers which are commonly used for dispersing anhydrous particles in an aqueous phase. The expression “free of emulsifying surfactants or of stabilizing polymers” is understood to mean compositions containing less than 0.1% of emulsifier and/or less than 0.05% of stabilizing polymer. The expression stabilizing polymer is understood here to mean a thickening polymer which does not have the property of forming a gel defined by the formation of a permanent three-dimensional macromolecular network.

By virtue of the method used, it is possible to prepare compositions containing fairly high polymer concentrations, which concentrations would be impossible to achieve if the destructured gel was first prepared from the polymer, that is to say a gel consisting of an infinite number of microparticles of polymer gel, and then mixed with an aqueous dispersion of particles of elastomeric silicone. Thus, the method used according to the present invention makes it possible to obtain compositions having a semisolid to pasty texture, and therefore constituting a paste which is solid to a greater or lesser degree. The expression “a semisolid to pasty texture” is understood here to mean a texture corresponding to a viscosity ranging from 100 000 (=10⁵) to 500 000 (=5·10⁵) Pa.s, this viscosity being measured at 25° C. with a Haake Rheostress RS 150 apparatus in a flat-cone configuration.

Thus, the subject of the present invention is also a method of preparing the composition as defined above, characterized in that it consists in mixing, at a temperature greater than the gelling temperature of the polymer, the polymer and the aqueous dispersion of particles of elastomeric organopolysiloxane, and the other optional additives, and then in cooling the mixture while shearing it.

It should be noted that this method of mixing in the hot state does not make it possible to obtain a homogeneous composition if pre-emulsified silicone dispersions as described in the document WO-A-99/51193 are used in place of the aqueous dispersion of elastomeric silicone, because the surfactants present in these pre-emulsified dispersions are in a relatively large quantity and are charged (anionic surfactants), which prevents gelling of the polymers. In the present invention, the amount of surfactants present are such that they do not prevent gelling of polymers; in addition, if they are present, they are generally nonionic, and therefore not charged.

The particles of elastomeric silicones of the invention are provided in the form of a homogeneous dispersion (or suspension) in water. The elastomeric silicones constituting the particles may be chosen in particular from the crosslinked polymers described in the document JP-A-10/175816. According to this document, they are obtained by addition and crosslinking reaction, in the presence of a catalyst, in particular of the platinum type, of at least:
(a) one organopolysiloxane (i) having at least two vinyl groups at the α,ω-position of the silicone chain per molecule; and
(b) one organosiloxane (ii) having at least one hydrogen atom linked to one silicon atom per molecule.

In particular, the organopolysiloxane (i) is chosen from polydimethylsiloxanes and is more specifically an α,ω-dimethylvinylpolydimethylsiloxane.

The aqueous dispersion of particles of elastomeric organopolysiloxane which is used in the composition according to the invention may be obtained in particular as follows:
(a) mixing the organopolysiloxane (i) and the organosiloxane (ii);
(b) adding an aqueous phase containing an emulsifier to the mixture of step (a);
(c) emulsifying the aqueous phase and the said mixture;
(d) adding hot water to the emulsion of step (c); and
(e) polymerizing the organopolysiloxane (i) and the organosiloxane (ii) in emulsion in the presence of a platinum catalyst.

Preferably, step (c) is effected in the presence of a nonionic emulsifier.

In this method, the water is advantageously added at a temperature of about 40 to 60° C. After step (e), it is possible to dry the particles obtained so as to evaporate therefrom some or all of the water trapped.

The organopolysiloxane particles obtained are deformable solid particles having a degree of hardness, which can be measured with a Shore A durometer (according to the ASTM D2240 standard) at room temperature or with the Japanese JIS-A method. This hardness may be measured on a block of elastomer prepared for this purpose as follows: mixing the organopolysiloxane (i) and the organosiloxane (ii); removal of air from the mixture; moulding and vulcanization in an oven at 100° C. for 30 minutes; cooling to room temperature and then measuring the hardness. The density is also determined on this elastomer block.

In particular, the Shore hardness of the particles used in the composition of the invention is preferably less than or equal to 80 Shore A, and even better less than or equal to 65 Shore A. The aqueous dispersions of elastomeric organopolysiloxanes used in the composition of the invention may be, for example, those marketed under the names BY 29-122 and BY 29-119 by the company Dow Corning. It is also possible to use a mixture of these commercial products. An elastomer block based on the product BY 29-122 has a hardness of 7 and based on the product BY 29-119 a hardness of 30. The density ranges from about 0.97 to 0.98. The dispersions of BY 29-122 and BY 29-119 contain about 63% by weight of particles of elastomeric organopolysiloxane (and therefore about 63% dry matter content) relative to the total weight of the dispersion. For example, BY 29-119 is an aqueous dispersion of particles of elastomeric organopolysiloxane comprising 36.35% of water, 63% of particles of elastomeric organopolysiloxane, 0.25% of C12-15 Pareth-12, 0.1% of sodium polyacrylate and 0.3% of phenoxyethanol (preservative).

In particular, the particles of elastomeric organopolysiloxane (as active substance) have a mean size in numerical terms ranging from 0.1 to 500 μm, and even better from 0.1 to 200 μm. These particles may be spherical, flat or amorphous, preferably with a spherical shape.

Thus, as indicated in the method described above, in order to stably disperse in water, these organopolysiloxane particles may be combined with one or more nonionic, cationic or anionic emulsifiers with an HLB (hydrophilic lipophilic balance) greater than or equal to 8. The proportion of emulsifiers in the aqueous dispersion preferably ranges from 0.1 to 20 parts by weight per 100 parts by weight of the aqueous dispersion of elastomeric organopolysiloxane, and even better from 0.5 to 10 parts by weight (see description of the document JP-A-10/175816).

In addition, these particles of elastomeric organopolysiloxane may be combined, in the aqueous dispersion, with fatty substances, in particular waxes or gums which are solid at room temperature, pasty fatty substances, of animal, plant, mineral or synthetic origin, mixtures thereof, and inorganic powders such as those described in this document.

Preferably, the particles of elastomeric organopolysiloxane are present in the composition of the invention in a quantity of active substance ranging, for example, from 0.1 to 25% by weight, preferably from 1 to 20% by weight, and even better from 5 to 15% by weight relative to the total weight of the composition.

The polymers used in the composition of the present invention are chosen from polymers capable of forming a thermoreversible gel, polymers capable of forming a crosslinked gel and mixtures thereof. According to a preferred embodiment of the invention, the polymer is chosen from polymers capable of forming thermoreversible gels. The expression “gel” is understood to mean a macroscopic three-dimensional network formed by macromolecules and giving the system thus formed viscoelastic properties defined by the fact that the elastic modulus is greater than the viscous modulus. This network is stabilized by bonds/interactions of the hydrogen, electrostatic and/or dispersive (=interactions between a polar compounds, for example between CH₂ groups) bond type. The expression “thermoreversible” is understood to mean that the gel state is obtained in a reversible manner, that is to say that the aqueous solution of polymer used is liquid at a temperature greater than the gelling temperature of the polymer and that, after cooling to a temperature less than the gelling temperature of the polymer, the solution solidifies. This gelling temperature depends on the polymer used.

The expression “crosslinked gel” is understood to mean a gel formed from a polymer solution to which there is added a crosslinking agent whose role is to physically connect the molecules of polymers so as to form a permanent three-dimensional network.

The polymer capable of forming a thermoreversible or alternatively a crosslinked gel is generally chosen from polymers of natural origin.

The polymers which may be used in the composition of the invention may be chosen in particular from carrageenans, agars, gellan gums, alginates, pectins, and mixtures thereof.

The carrageenans, the agars and the gellan gums are polymers capable of forming a thermoreversible gel. They should be in a sufficient quantity in order to behave like polymers capable of forming a thermoreversible gel, and not like thickening polymers forming a rheofluidizing gel. Thus, the quantity is generally greater than or equal to 3% of the total weight of the composition.

The carrageenans are linear polysaccharides present in certain red algae. They consist of alternating β-1,3- and α-1,4-galactose residues, it being possible for many galactose residues to be sulphated. This polysaccharide family is described in Chapter 3 of the book “Food Gels” published by Peter Harris, Elsevier 1989. As carrageenans, there may be mentioned, for example, kappa carrageenan, iota carrageenan and mixtures thereof. It is also possible to use mixtures of these carrageenans with lambda-carrageenan which does not form gels in aqueous solution but which may exist as a mixture with the carrageenans mentioned above which, for their part, form gels. Preferably, kappa-carrageenan is used as carrageenan.

Agars are polymers extracted from certain red algae, which contain agarose and agaropectin, polysaccharides comprising alternating α-1,4-galactose residues and β-1,3-galactose residues, with sulphate, pyruvate and/or glucuronic residues in the case of agaropectin. Agars are described in Chapter 1 of the book “Food Gels” mentioned above. As agars, there may be mentioned, for example, the product marketed under the name Agar Luxara 1253 by the company Arthur Bramwell.

Gellan gums are polymers of bacterial origin, which are polysaccharides consisting of alternating glucose, glucuronic acid and rhamnose residues. The polymers belonging to the gellan family are described in Chapter 6 of the- book “Food Gels” mentioned above. As gellan gums, there may be mentioned, for example, the product marketed under the name Kelcogel F by the company Nutrasweet Kelco.

Alginates and pectins are polymers forming gels of the crosslinked type; the formation of the gel is induced therein by the addition of salts acting as crosslinking agents. Alginates are products consisting of polysaccharide chains obtained by the linkage of two types of monomer, glucuronic acid (G) and mannuronic acid (M) which are organized into G blocks, M blocks and blocks with alternating G-M monomers. Gelling is obtained by bridging of these polysaccharide chains with the aid of polyvalent ions. This bridging preferably occurs between the G blocks. As alginate, there may be mentioned, for example, sodium alginate sold under the trade name Manugel DMB by the company Nutrasweet Kelco and partial alkyl or hydroxyalkyl alginates such as propylene glycol alginate sold by the company Kelco under the name Kelcoloid O, which is a propylene glycol alginate having a degree of esterification of 80 to 86%.

As pectin, there may be mentioned, for example, the product marketed under the name Rapid Set Pectin by the company Unipectine.

It is possible to use one or more of these polymers capable of forming a thermoreversible or crosslinked gel. The quantity of polymer(s) capable of forming a thermoreversible or crosslinked gel in the composition of the invention may range, for example, from 3 to 15% by weight, and preferably from 4 to 10% by weight relative to the total weight of the composition.

It is optionally possible to add to the polymer(s) capable of forming a thermoreversible or crosslinked gel one or more water-soluble polymers forming a gel, as described above, insofar as they are in a small quantity and do not alter the desired properties of the composition according to the invention. As water-soluble polymers, there may be mentioned, for example, xanthan gum, cellulosic polymers, and polymers derived from acrylamide, in particular homopolymers or copolymers containing at least one monomer having an ethylenic unsaturation and having a sulphonic group, which may be in free form or partially or completely neutralized, more particularly homopolymers or copolymers of polyacrylamidomethylpropanesulphonic acid (AMPS), such as the polymer marketed under the name Hostacerin AMPS by the company Clariant. When these water-soluble polymers are present, they are preferably in a quantity less than 0.1%, preferably less than 0.05% by weight, and even better less than 0.02% by weight relative to the total weight of the composition. The quantity of water-soluble polymer may range, for example, from 0.0001 to 0.1% by weight, preferably from 0.0005 to 0.05% by weight, and even better from 0.0005 to 0.02% by weight relative to the total weight of the composition.

The cellulosic polymers may be chosen, for example, from cellulose or its derivatives such as carboxymethylcellulose, hydroxypropylcellulose, methylcellulose, hydroxypropylmethylcellulose, hydroxyethylcellulose, modified celluloses, in particular modified by grafting an alkyl group, and mixtures thereof.

The composition according to the invention comprises a quantity of water generally ranging from 60 to 97%, preferably from 75 to 95%, and even better from 80 to 90% by weight relative to the total weight of the composition.

The composition according to the invention is appropriate for topical use and it can constitute in particular a cosmetic or dermatological composition.

As indicated above, the preparation of the composition of the invention may be carried out in a single step in the hot state. The expression “a single step” is understood to mean that the aqueous dispersion of particles of elastomeric organopolysiloxane and the polymer are mixed in the hot state before gelling of the polymer, and therefore that there is no prior preparation of the gel. Thus, this method comprises in mixing, at a temperature greater than the gelling temperature of the polymer (for example 90° C.), the polymer and aqueous dispersion of the particles of elastomeric organopolysiloxane, and the other optional additives, and then in cooling the mixture while shearing it (vigorous stirring).

This method makes it possible to be able to prepare a composition containing a large quantity of polymer, and thus to be able to obtain a paste consisting of the homogeneous mixture of a polymer gel and of silicone particles. Because of this, as indicated above, it is possible to obtain aqueous and pasty cosmetic compositions containing silicone powders, while being free or practically free of surfactants and/or of stabilizing polymers, in which the elastomeric silicone powders preserve their properties.

In more detail, the method may comprise the following steps:
preparation of an aqueous solution by heating the aqueous dispersion of particles of elastomeric organopolysiloxane to a temperature greater than the gelling temperature of the polymer (for example 90° C.), and mixing the heated dispersion with the polymer, this mixing preferably being carried out with slow stirring (30 to 100 revolutions/minute), with optional addition of heated water (at 90° C.), in order to adjust the relative concentrations of polymer and of particles of elastomeric organopolysiloxane;
stirring the solution (gentle stirring of 30 to 100 rpm) while maintaining the temperature, in order to ensure complete solubilization (temperature of about 90° C.);
cooling the solution to room temperature while subjecting it to shearing in order to obtain a paste (destructuring phase); the shearing rate and time should be sufficient in order to obtain a paste (rate of about 2000 to 4000 rpm for about 60 minutes);
stirring (gentle stirring: about 30 to 100 rpm) of the paste at room temperature to homogeneity (stirring for about 1 hour).

By virtue of this method, a paste is obtained which consists of a homogeneous mixture of a gel of polymer and of silicone powders which is crosslinked. Examination under a microscope of the compositions obtained shows that the silicone particles preserve their structural integrity throughout implementation. Thus, the sensory properties, and in particular the softness, which are associated with the use of these particles are preserved.

The method described above may be carried out in any appropriate apparatus and, for example, in a tank equipped with a propeller and with blades, such as an OLSA® pilot mixer, or in a twin-screw extruder.

In the case of the polymers forming crosslinked-type gels, it is possible to use the method described above provided the crosslinking agent is injected during the phase of destructuring the polymer solution or alternatively, if the method is carried out in a twin-screw extruder, during extrusion of the polymer solution.

The compositions according to the invention possess characteristic rheological parameters comprising the yield point, the elastic modulus and the viscous modulus. The destructuring yield stress or yield point τ₀ of the compositions is defined as the pressure required to cause a macroscopic flow of the composition; it can be determined by scanning under stress, for example with the aid of an applied stress Haake rheometer CS150 at a temperature of 25° C.

Moreover, the elastic modulus and the viscous modulus of the compositions may be measured for a stress frequency of 1 Hertz, in the so-called zone of linear viscoelasticity defined by the fact that the stress applied during the measurement is less than the destructuring yield stress of the composition.

The compositions according to the invention are pasty and are characterized by an elastic modulus, at 25° C., greater than 5000 Pascals, and preferably of 10 000 Pascals, and a tangent value (δ), given by the ratio of the viscous modulus to the elastic modulus, of less than 0.2, at 25° C. for a stress frequency of 1 Hertz. The yield point of the compositions according to the invention is greater than 50 Pascals or of the order of 100 Pascals. The viscosity of the compositions according to the invention is greater than or equal to 100 000 Pa.s (=10⁵ Pa.s) and is preferably greater than or equal to 300 000 Pa.s (=3.10⁵ Pa.s), the viscosity being measured at 25° C. with a Haake Rheostress RS 150 apparatus in a flat-cone configuration under a stress of about 100 Pascals.

The rheological parameters of the composition of the example illustrating the invention are given after the example.

As indicated above, the composition according to the invention is preferably pasty and constitutes a paste which is solid to a greater or lesser degree, that is to say which may have a semisolid to pasty, and even solid, texture.

In a known manner, the compositions of the invention may contain one or more adjuvants chosen from the adjuvants which are customary in the cosmetic and dermatological fields, such as hydrophilic or lipophilic active agents, preservatives, antioxidants, perfumes, fillers, colouring matter (pigments or colourants), lipid vesicles, and mixtures thereof. These adjuvants are used in the proportions which are customary in the cosmetic or dermatological field, and for example of from 0.01 to 30% of the total weight of the composition.

Of course, persons skilled in the art will be careful to choose this or these optional additional compounds and/or their quantity such that the advantageous properties of the composition according to the invention are not, or not substantially, altered by the addition envisaged.

As active agents, there may be mentioned in particular sunscreens; moisturizing agents such as polyols (glycerin, glycols); vitamins and in particular vitamins A (retinol), C (ascorbic acid), E (tocopherol), B3 (niacinamide), B5 (panthenol), H (biotin), F and D and their derivatives and in particular their esters; keratolytic or prodesquamatory agents, for example α-hydroxy acids; β-hydroxy acids; α-keto acids; β-keto acids; soothing agents such as alpha-bisabolol, allantoin and gamma-oryzanol; urea; rutin; enzymes; natural extracts such as green tea, melissa extract, thyme extract, procyanidolic oligomers (PCO) such as hawthorn PCO, pine PCO and grape PCO; certain acids such as kbjic acid, caffeic acid, retinoic acid and its derivatives, 1,4-benzene[di(3-methylidene-10-camphorsulphonic)] acid; carotenoids such as carotenes such as for example α, β- and γ-carotenes, β,ψ-carotene, ε-carotene, β,λ-carotene, lycopene (Ψ,Ψ-carotene); and mixtures thereof.

As fillers which may be used in the composition of the invention, there may be mentioned, for example, polyamide particles and in particular those sold under the name Orgasol by the company Atochem; polyethylene powders; microspheres based on acrylic copolymers, such as those made of ethylene glycol dimethacrylate/lauryl methacrylate copolymer which are sold by the company Dow Corning under the name Polytrap; expanded powders such as hollow microspheres, and in particular the microspheres marketed under the trade name Expancel by the company Kemanord Plast or under the trade name Micropearl F 80 ED by the company Matsumoto; powders of natural organic materials such as maize, wheat or rice starches, crosslinked or otherwise, such as starch powders crosslinked with octenylsuccinic anhydride, which are marketed under the name Dry-Flo by the company National Starch; microbeads of silicone resin such as those marketed under the name Tospearl by the company Toshiba Silicone; fibres; and mixtures thereof.

More particularly, when the filler is a fibre, the latter may be chosen from hydrophilic or hydrophobic fibres of synthetic or natural, inorganic or organic origin. These fibres may be short or long, individual or organized, for example plaited. They can have any shape or morphology, and in particular can have a circular or polygonal (square, hexagonal or octagonal) cross section, according to the specific application envisaged. In particular, their ends are blunt and/or smooth in order to prevent injury.

In particular, the fibres may have a length (L) ranging from 1 μm (0.001 mm) to 10 mm, preferably from 0.1 μm to 5 mm, and even better from 0.1 mm to 1.5 mm. Their cross section may be within a circle having a diameter (D) ranging from 1 nm (0.001 μm) to 100 μm, preferably ranging from 1 nm (0.001 μm) to 50 μm, and even better from 5 μm to 40 μm, with, preferably, an aspect ratio, that is to say an L/D (length/diameter) ratio ranging from 3.5 to 2500, even better from 5 to 500, and better still from 5 to 150, and a linear density ranging from 0.15 to 30 denier, and even better from 0.18 to 18 denier, the denier being the weight in grams for 9 km of thread.

The fibres may be those used in the manufacture of textiles, and in particular silk fibres, cotton fibres, wool fibres, flax fibres, cellulose fibres which are extracted in particular from wood, vegetables or algae, polyamide fibres (Nylon®), modified cellulose fibres (rayon, viscose, acetate, in particular rayon acetate), poly(p-phenylene terephthalamide) fibres, in particular Kevlar®, acrylic fibres, in particular poly(methyl methacrylate) or poly(2-hydroxyethyl methacrylate) fibres, polyolefin fibres, and in particular polyethylene or polypropylene fibres, glass fibres, silica fibres, aramid fibres, carbon fibres, in particular in graphite form, Teflon® fibres, insoluble collagen fibres, polyester fibres, poly(vinyl chloride) or poly(vinylidene chloride) fibres, poly(vinyl alcohol) fibres, polyacrylonitrile fibres, chitosan fibres, polyurethane fibres or poly(ethylene phthalate) fibres, fibres formed from a mixture of polymers such as those mentioned above, such as polyamide/polyester fibres, and mixtures of these fibres.

As polyurethane fibres, there may be mentioned the fibres made of segmented poly(urethane urea) polymer, belonging to the class of elastanes, and in particular those marketed under the name Lycra® by the company Du Pont.

It is also possible to use the resorbable synthetic fibres used in surgery, such as the fibres prepared from glycolic acid and caprolactone (Monocryl from the company Johnson & Johnson); resorbable synthetic fibres of the copolymer of lactic acid and glycolic acid type (Vicryl from the company Johnson & Johnson); terephthalic polyester fibres (Ethibond from the company Johnson & Johnson) and stainless steel wires (Acier from the company Johnson & Johnson).

Moreover, the fibres may or may not be surface-treated and may or may not be coated. They may be in particular coated and/or functionalized fibres, “functionalized” meaning that the fibres are surface-treated so as to modify the properties thereof.

As coated fibres which can be used in the invention, there may be mentioned polyamide fibres coated with copper sulphide for an antistatic effect (for example R-STAT from the company Rhodia) or another polymer allowing a particular organization of the fibres (specific surface treatment) or a surface treatment which induces colour/hologram effects (Lurex fibre from the company Sildorex, for example).

The fibres may also be functionalized, that is to say may be modified so as to have a specific function. This functionalization of the fibres may be carried out both on the fibres and in the fibres, this being by any method which makes it possible to attach a compound to the fibres or to trap it in the cavities formed by the geometry of the fibres. As methods, there may be mentioned, for example, the coating of fibres with an active agent; the attachment of particles containing an active agent, such as nanocapsules or nanospheres, onto the fibres; adsorption in the fibres; attachment by chemical reaction. It is thus possible to use fibres having specific functionalities, for example anti-UV fibres by modification with chemical or physical sunscreening agents; bactericidal or antiseptic fibres by modification with preservatives or antibacterial agents; coloured fibres by modification with colouring molecules; keratolytic or desquamatory fibres by modification with keratolytic or desquamatory agents; moisturizing fibres by modification with moisturizing agents or water-retaining polymers; perfumed fibres by modification with a perfume; analgesic or soothing fibres by modification with an anti-inflammatory agent or a soothing agent; antiperspirant fibres by modification with an antiperspirant.

As fibres, there may be mentioned more especially polyamide fibres, poly(p-phenyleneterephthalamide) fibres, cotton fibres, and mixtures thereof. Their length may range from 0.1 to 10 mm, preferably from 0.1 to 1 mm, their mean diameter may range from 5 to 50 μm and the aspect ratio preferably ranges from 5 to 150.

In particular, it is possible to use polyamide fibres marketed by Etablissements P. Bonte under the name Polyamide 0.9 dtex 0.3 mm, having a mean diameter of 15 to 20 μm, a linear density of about 0.9 dtex (0.81 denier) and a length ranging from 0.3 mm to 1.5 mm; poly(p-phenyleneterephthalamide) fibres having a mean diameter of 12 μm and a length of about 1.5 mm such as those sold under the name Kevlar Floc by the company Du Pont Fibres; cotton fibres, for example those having a mean diameter of 20 μm, a length of 0.3 mm, and an aspect ratio of 15, such as those marketed by Institut Textile de France, the company Filature de Lomme, by the company Textiles des Dunes or by the company Velifil.

The quantity of fillers may range from 0.01 to 20% by weight, and preferably from 0.1 to 10% by weight of active substance relative to the total weight of the composition.

The compositions which are the subject of the invention find application in particular in a large number of cosmetic treatments of the skin and the lips, including the scalp, in particular for the treatment, protection and care of the skin and/or the lips, and more particularly for softening, correcting and/or concealing imperfections of the skin and/or the lips, such as microreliefs, wrinkles, fine lines, pores or variations in colour.

Accordingly, the subject of the invention is also the cosmetic use of the composition as defined above, for the treatment, protection and/or care of the skin and/or the lips.

The subject of the invention is also the cosmetic use of the composition as defined above for softening, correcting and/or concealing imperfections of the skin and/or the lips.

The subject of the invention is also a method for the cosmetic treatment of the skin, including the scalp, and/or of the lips, characterized in that a composition as defined above is applied to the skin and/or the lips.

The compositions of the invention may also be intended for the treatment of dry skin and/or dry lips and/or sensitive skin.

The subject of the invention is also the use of the composition as defined above for the manufacture of a composition intended for the care of dry skin and/or dry lips and/or sensitive skin.

The example of composition given below is given by way of illustration and without limitation. The quantities are given as a percentage by weight, unless otherwise stated.

Example: Gel

| | |
|---|---|
| Agar (Agar Luxara 1253) | 5.5% |
| Aqueous dispersion of silicone powder (containing | 16.6% |
| 63.5% active substance of particles of elastomeric | |
| organopolysiloxane (BY 29-119) | |
| Preservatives | 0.25% |
| Water | 77.65% |

A gel is obtained which is cooling during application, which is very soft and which makes it possible to correct imperfections of the skin microrelief.

The Theological parameters of this composition at 25° C., under the conditions indicated above, are presented in the following table:

| | Example of the invention |
|---|---|
| Elastic modulus G′ at 1 Hertz (Pa) | 11200 |
| Viscous modulus G″ at 1 Hertz (Pa) | 1200 |
| Yield point, τ₀ (Pa) | 120 |
| Viscosity (Pa · s)* | 352000 |
| | (3.52 · 10⁵) |

*measured under a stress of about 100 Pascals.

1. A composition for topical application to the skin or the lips comprising, in a physiologically acceptable medium,
(a) at least one polymer selected from the group consisting of polymers capable of forming a thermoreversible gel, polymers capable of forming a crosslinked gel, and mixtures thereof, and
(b) an aqueous dispersion of particles of elastomeric organopolysiloxane,
wherein the elastomeric organopolysiloxane is obtained by addition and crosslinking reaction, in the presence of a catalyst, of at least:
one organopolysiloxane (i) having two vinyl groups at the α,ω-position of the silicone chain per molecule; and
one organosiloxane (ii) having at least one hydrogen atom linked to one silicon atom per molecule.

2. The composition according to claim 1, wherein the organopolysiloxane (i) is chosen from polydimethylsiloxanes.

3. The composition according to claim 1, wherein the organopolysiloxane (i) is an α,ω-dimethylvinylpolydimethylsiloxane.

4. The composition according to claim 1, wherein the aqueous dispersion of organopolysiloxane particles is obtained by a processing, which comprises:
(a) mixing the organopolysiloxane (i) and the organosiloxane (ii);
(b) adding an aqueous phase comprising an emulsifier to the mixture of step (a);
(c) emulsifying the aqueous phase and the mixture to obtain an emulsion;
(d) adding hot water to the emulsion; and
(e) polymerizing the organopolysiloxane (i) and the organosiloxane (ii) in emulsion in the presence of a platinum catalyst.

5. The composition according to claim 4, wherein said emulsifying is carried out in the presence of a nonionic emulsifier.

6. The composition according to claim 1, wherein the organosiloxane particles have a mean size ranging from 0.1 to 500 μm.

7. The composition according to claim 1, wherein the organopolysiloxane particles have a hardness of less than or equal to 80 Shore A.

8. The composition according to claim 1, wherein the organosiloxane particles are present in a quantity of active substance ranging from 0.1 to 25% by weight relative to the total weight of the composition.

9. The composition according to claim 1, wherein the polymer is selected from the group consisting of carrageenans, agars, gellan gums, alginates, pectins, and mixtures thereof.

10. The composition according to claim 1, wherein the quantity of polymer ranges from 3 to 15%, by weight relative to the total weight of the composition.

11. The composition according to claim 1, wherein the composition comprises from 60 to 97% of water, by weight relative to the total weight of the composition.

12. The composition according to claim 1, wherein the composition has, at 25° C., a viscosity greater than or equal to 10⁵ Pa.s.

13. The composition according to claim 1, wherein the composition comprises less than 0.1% by weight of emulsifier.

14. The composition according to claim 1, wherein the composition comprises less than 0.05% by weight of a stabilizing polymer.

15. The composition according to claim 1, wherein the composition is in the form of a a pasty composition.

16. The composition according to claim 1, wherein the composition is in the form of a cosmetic or dermatological composition.

17. A method of preparing the composition according to claim 1, the method comprising:
mixing, at a temperature greater than the gelling temperature of the polymer, the polymer and the aqueous dispersion of particles of elastomeric organopolysiloxane, and the other optional additives to obtain a mixture, and then
cooling the mixture while shearing the mixture.

18. The method according to claim 17, wherein
said mixing comprises:
preparing an aqueous solution by heating the aqueous dispersion of particles of elastomeric organopolysiloxane to a temperature greater than the gelling temperature of the polymer,
mixing the heated dispersion with the polymer, with optional addition of heated water, to adjust the relative concentrations of polymer and of particles of elastomeric organopolysiloxane;
stirring the solution while maintaining the temperature, in order to ensure complete solubilization;
and said cooling comprises:
cooling the solution to room temperature while shearing to obtain a paste; and
the method further comprises
stirring the paste at room temperature to homogeneity.

19. Cosmetic A method for the protection and/or care of the skin and/or the lips, the method comprising:
applying the composition according to claim 1 to the skin and/or the lips in need thereof.

20. A method for softening, correcting and/or concealing imperfections of the skin and/or the lips, the method comprising:
applying the composition according to claim 1 to the skin and/or lips in need thereof.

21. A method for the cosmetic treatment of the skin and/or the lips, the method comprising:
applying the composition according to claim 1 to the skin and/or the lips.

22. A method for the manufacture of a composition intended for the care of dry skin and/or dry lips and/or sensitive skin comprising:
incorporating the composition as claimed in claim 1 to the composition intended for the care of dry skin and/or dry lips and/or sensitive skin.
